(19) United States
(12) Patent Application Publication
(10) Pub. No.: US 2024/0142452 A1
(43) Pub. Date: May 2, 2024

(54) Non-Invasive Method and System for Classifying a Liver Condition in a Human Subject

(71) Applicant: Fibronostics. Inc., Orlando, FL (US)

(72) Inventor: Carmen Mona MUNTEANU, PARIS (FR)

(21) Appl. No.: 17/976,988

(22) Filed: Oct. 31, 2022

(51) Int. Cl. G01N 33/573; G01N 33/66; G01N 33/72; G01N 33/92; G16H 50/20

(52) U.S. Cl. CPC G01N 33/573; G01N 33/66; G01N 33/728; G01N 33/92; G16H 50/20; G01N 2800/085; G01N 2800/56; G01N 2800/60

(57) ABSTRACT

Identifying and classifying a liver condition in a subject based on a blood sample obtained from the subject in a system functionally associated with a blood sample analyzer. The system includes an input interface or a transceiver, a processors, a database, and a computer readable storage medium for instructions execution by the processor(s). The storage medium stores instructions to receive information relating to the subject, and instructions to receive measurements of a plurality of serum biomarkers. The storage medium further stores instructions to identify a group to which the subject belongs, and to obtain from the database average or standardized biomarker measurements for the group. The storage medium further stores instructions to apply a neural network algorithm to the received measurements and biographical information, and instructions to identify, based on an output of the neural network algorithm, the presence of a liver condition, and to classify a severity of the liver condition.

### FIELD OF THE DISCLOSED TECHNOLOGY

The disclosed technology relates generally to methods and systems for diagnosing a liver condition in a human subject, and, more specifically, to non-invasive methods and systems for identifying the presence of a liver condition, and/or classifying the severity of such liver condition, using a blood sample and anthropometric data.

### BACKGROUND OF THE DISCLOSED TECHNOLOGY

Chronic liver diseases, such as chronic viral hepatitis, non-alcoholic fatty liver diseases (NAFLD), and non-alcoholic steatohepatitis (NASH), usually develop over many years. These conditions are leading causes of morbidity and mortality globally, and the prevalence thereof has been increasing in recent years due to obesity and diabetes. NAFLD now represents the most common cause of abnormal liver blood tests but usually causes no signs or symptoms and is the most common liver disease in the Western world. NASH is currently the second leading cause of liver disease among those waiting for liver transplants in the US.

Percutaneous liver biopsy remains the gold standard for precisely diagnosing NAFLD, specifying the category (severity) thereof. A biopsy is also necessary to assess the histopathologic criteria essential to diagnosis of NAFLD/NASH. Biopsies further allow for confirmation of steatosis, as well as determining a degree of lobular inflammation, ballooning, and fibrosis. However, such biopsies are invasive, and include an intrinsic risk factor to the procedure.

The severity of NAFLD is typically evaluated based on a scoring system, such as the NASH-CRN, which evaluates, and assigns scores to, four domains: steatosis (scores in the range of 0-3), lobular inflammation (scores in the range of 0-3), hepatocyte ballooning (scores in the range of 0-2), and liver fibrosis (scores in the range of 0-4). In some cases, the first three scores are summed to generate an aggregate value (composite activity score), whereas the fibrosis staging score is maintained separate.

NASH is typically diagnosed based on an overall assessment of the biopsied tissue by a pathologist, using scoring systems such as the Steatosis, Activity, and Fibrosis (SAF) score. A simplified histopathology score, the SAF score of Bedossa et al., evaluates the presence and extent of each individual component of steatosis, activity (based on lobular inflammation and ballooning) and fibrosis.

Some non-invasive methods have been disclosed for evaluating non-alcoholic fatty liver diseases-related fibrosis and/or steatosis in a non-invasive and automated manner. However, some such prior art methods require information relating to biomarkers which are not routinely checked, and may be complex to obtain.

There is thus a need in the art for a non-invasive method of automatically screening at-risk subjects for NAFLD presumedfeatures [fibrosis, steatosis] using only commonly used biomarkers as well as ruling out presumed disease in those at-risk subjects with normal livers.

### SUMMARY OF THE DISCLOSED TECHNOLOGY

The present disclosure relates to a method and a system for non-invasively screening to determine a presumed clinical category of a liver condition.

In accordance with an embodiment of the disclosed technology, there is provided a method of identifying and classifying a liver condition in a human subject, the method including obtaining subject-specific information relating to the human subject, the subject-specific information including at least age, sex, height, and weight. The method further includes using a plurality of analyzers, obtaining from serum or plasma in a blood sample obtained from the human subject measurements of a plurality of serum biomarkers, the plurality of serum biomarkers including at least two biomarkers selected from the group consisting of total Bilirubin, gamma-glutamyl transpeptidase (GGT), alanine-aminotransferase (ALT), aspartate aminotransferase (AST), Total cholesterol, fasting triglycerides, and fasting glucose. Based on the obtained subject-specific information, a population to which the human subject belongs is identified, and average measurements of a second plurality of biomarkers for the population are obtained from a database, the second plurality of biomarkers selected from the second group consisting of Alpha-2-Macroglobulin, Apolipoprotein A1, Haptoglobin and platelets count. Using a processor executing instructions stored in a non-transitory computer memory, a neural network algorithm is applied to the measurements of the plurality of biomarkers, the average measurements, and the subject-specific information, and based on an output of the neural network algorithm, the presence of a liver condition is identified and a severity of the liver condition is classified.

In some embodiments, the plurality of serum biomarkers excludes Alpha-2-Macroglobulin, Apolipoprotein A1, Haptoglobin and platelets count.

In some embodiments, at least one test tube used for obtaining the blood sample includes at least one of a test tube containing lithium heparin, a test tube containing a glycolytic inhibitor, a test tube containing sodium fluoride, and a test tube containing potassium oxalate.

In some embodiments, the output of the neural network algorithm includes a classification into one of a plurality of stages, each stage indicating the presence or absence of the liver condition, and the severity of the liver condition.

In some embodiments, the method further includes, prior to the applying the neural network algorithm, pre-processing at least some of the measurements of the plurality of serum biomarkers or at least one data item of the subject-specific information. In some embodiments, the pre-processing includes logarithmically scaling at least some of the measurements of the plurality of serum biomarkers. In some embodiments, the pre-processing includes standardizing at least some of the measurements of the plurality of serum biomarkers and at least one data item of the biographical information.

In some embodiments, the obtained subject-specific information additionally includes at least one of nationality, ethnicity, area of residence, and medical history of the subject.

In some embodiments, the method further includes, further including, based on the classification of the liver condition, evaluating the risk of the subject developing a severe outcome to a viral infection. In some embodiments, the viral infection includes a COVID-19 infection.

In accordance with another embodiment of the disclosed technology, there is provided a system of identifying and classifying a liver condition in a human subject based on a blood sample obtained from the human subject, the system being functionally associated with at least one analyzer for analyzing the blood sample. The system includes at least one of an input interface or a transceiver, a database storing anthropometric and/or medical data relating to a plurality of subjects, one or more processors functionally associated with the at least one input interface or transceiver and with the database, and a non-transitory computer readable storage medium for instructions execution by the one or more processors. The non-transitory computer readable storage medium has stored: (i) instructions to receive subject-specific information relating to the human subject, the subject-specific information including at least age, gender, height, and weight; (ii) instructions to receive, from the at least one analyzer, measurements of a plurality of serum biomarkers, the plurality of serum biomarkers including at least two biomarkers selected from the group consisting of total Bilirubin, gamma-glutamyl transpeptidase (GGT), alanine-aminotransferase (ALT), aspartate aminotransferase (AST), Total fasting cholesterol, fasting triglycerides, and fasting glucose; (iii) instructions to identify, based on the received subject-specific information, a population to which the human subject belongs; (iv) instructions to obtain from the database average measurements of a second plurality of biomarkers for the population, the second plurality of biomarkers selected from the second group consisting of Alpha-2-Macroglobulin, Apolipoprotein A1, Haptoglobin and platelets count; (v) instructions to apply a neural network algorithm to the measurements of the plurality of biomarkers, the average measurements, and the subject-specific information; and (vi) instructions to identify, based on an output of the neural network algorithm, the presence of a liver condition, and to classify a severity of the liver condition.

In some embodiments, the plurality of serum biomarkers excludes Alpha-2-Macroglobulin, Apolipoprotein A1, Haptoglobin, and platelets count.

In some embodiments, the non-transitory computer readable storage medium further has stored instructions, to be executed prior to execution of the instructions to applying the neural network algorithm, to pre-process at least some of the measurements of the plurality of serum biomarkers or at least one data item of the subject-specific information. In some embodiments, the instructions to pre-process include instructions to logarithmically scale at least some of the measurements of the plurality of serum biomarkers. In some embodiments, the instructions to pre-process include instructions to standardize at least some of the measurements of the plurality of serum biomarkers and at least one data item of the subject-specific information.

In some embodiments, the instructions to identify the presence of a liver condition and to classify the severity of the liver condition include instruction to classify the subject into one of a plurality of stages, each stage indicating the presence or absence of the liver condition, and the severity of the liver condition.

In some embodiments, the subject-specific information additionally includes at least one of nationality, ethnicity, area of residence, and medical history of the subject.

In some embodiments, the non-transitory computer readable storage medium further has stored instructions to evaluate the risk of the subject developing a severe outcome to a viral infection based on the classification of the liver condition.

### BRIEF DESCRIPTION OF THE DRAWINGS

FIG. 1 is a schematic illustration of a system for identifying a clinical category of a liver disease, according to an embodiment of the disclosed technology.

FIG. 2 is a flow-chart of a method for identifying the presence of a liver condition, and classifying a severity thereof, according to an embodiment of the disclosed technology.

FIG. 3 shows a high-level block diagram of a device that may be used to carry out the disclosed technology.

### DETAILED DESCRIPTION OF EMBODIMENTS OF THE DISCLOSED TECHNOLOGY

In an embodiment of the disclosed technology, a system of identifying and classifying a liver condition in a human subject based on a blood sample obtained from the human subject is provided. The system is functionally associated with at least one analyzer for analyzing the blood sample. The system includes at least one of an input interface or a transceiver, a database one or more processors, and a non-transitory computer readable storage medium. The database stores anthropometric and/or medical data relating to a plurality of subjects. The processor(s) is functionally associated with the at least one input interface or transceiver and with the database. The storage medium stores instructions to be executed by the processor(s). The non-transitory computer readable storage medium has stored:
(i) instructions to receive subject-specific information relating to the human subject, the subject-specific information including at least age, gender, height, and weight;
(ii) instructions to receive, from the at least one analyzer, measurements of a plurality of serum biomarkers, the plurality of serum biomarkers including at least three biomarkers selected from the group consisting of total Bilirubin, gamma-glutamyl transpeptidase (GGT), alanine-aminotransferase (ALT), aspartate aminotransferase (AST), Total fasting cholesterol, fasting triglycerides, and fasting glucose;
(iii) instructions to identify, based on the received subject-specific information, a population to which the human subject belongs;
(iv) instructions to obtain from the database average measurements of a second plurality of biomarkers for the population, the second plurality of biomarkers selected from the second group consisting of Alpha-2-Macroglobulin, Apolipoprotein A1, Haptoglobin and platelets count;
(v) instructions to apply a neural network algorithm to the measurements of the plurality of biomarkers, the average measurements, and the subject-specific information; and
(vi) instructions to identify, based on an output of the neural network algorithm, the presence of a liver condition, and to classify a severity of the liver condition.

Embodiments of the disclosed technology will become clearer in view of the following description of the drawings.

In the context of the present specification and claims, the term “Fibrosis of the liver” is defined as formation of presumed excess fibrous connective tissue in the liver.

In the context of the present specification and claims, the term “Activity of the liver” is defined as presumed inflammation of the liver or hepatic necroinflammatory activity.

In the context of the present specification and claims, the term “Steatosis of the liver” is defined as presumed abnormal retention of fat.

In the context of the present specification and claims, the term “approximately” is defined as being within 10% of a target number or measure.

It should be understood that the use of “and/or” is defined inclusively such that the term “a and/or b” should be read to include the sets: “a and b,” “a or b,” “a,” “b.”

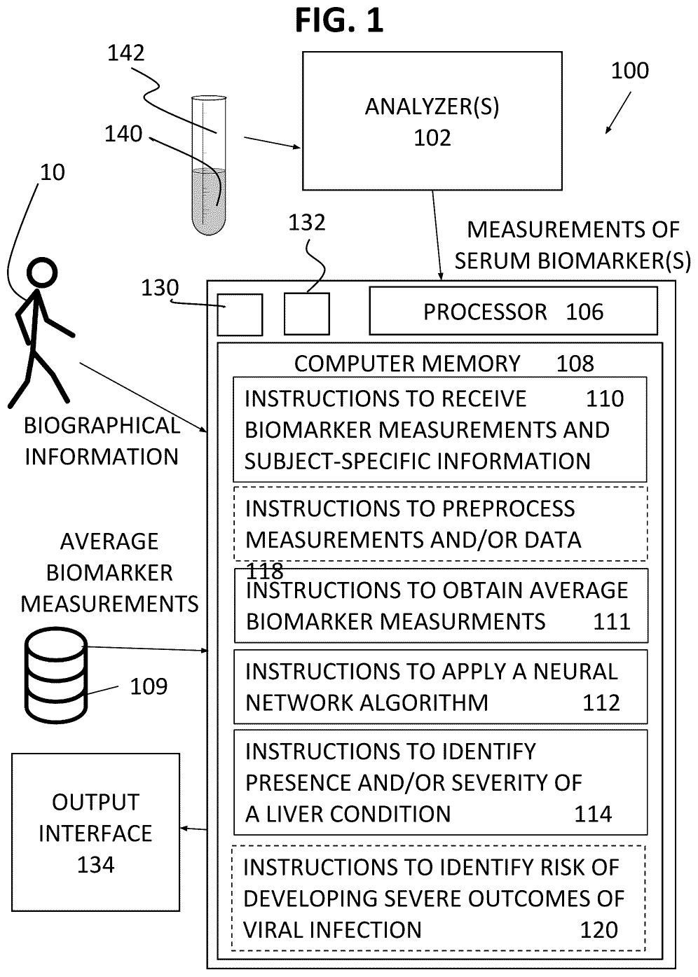

Reference is now made to FIG. 1, which is a schematic illustration of a system for classifying a presumed clinical category of non-alcoholic fatty liver disease (NAFLD) and/or steatohepatitis (NASH) in human subjects, according to an embodiment of the disclosed technology. As seen, a system 100 according to the disclosed technology comprises a computing device, functionally associated with a plurality of analyzers 102. The system 100 includes one or more processors 106, and a non-transitory computer readable storage medium 108, which stores instructions to be executed by the processor(s) 106. Processor(s) 106 is further associated with one or more databases 109, storing anthropometric and medical data relating to a plurality of subjects. For example, databases 109 may include data relating to biographical information of the plurality of subjects, racial information of the plurality of subjects, and medical information of the plurality of subjects, in particular medical information related to liver conditions and diseases.

Computer readable storage medium 108 has stored:
a) instructions 110 to receive, for example from analyzers 102, measurements of serum biomarkers obtained from blood of a human subject 10, as well as subject-specific information of the human subject 10;
b) instructions 111 to obtain, from database(s) 109, one or more average measurements of biomarkers of a population to which the human subject 10 belongs;
c) instructions 112 to apply a neural network algorithm to the measurements of serum biomarkers of the human subject and to the average biomarkers of the population to which the human subject belongs; and
d) instructions 114 to identify, based on the output of the neural network algorithm, the presence or absence of a liver condition, and if a liver condition is present, to identify a stage of the liver condition.

In some embodiments, computer readable storage medium 108 further has stored instructions 118 to scale at least some of the measurements of serum biomarkers, and/or instructions to standardize at least some of the measurements of serum biomarkers and/or at least some of the biographical information.

In some embodiments, computer readable storage medium 108 further has stored instructions 120 to identify, based on the identified stage of the liver condition, a risk of the subject developing severe outcomes of viral infections, such as COVID-19.

The instructions 110 to receive measurements of serum biomarkers include instructions to receive measurements of at least three, at least four, at least five, or all of the following biomarkers:
a) total Bilirubin;
b) gamma-glutamyl transpeptidase (GGT);
c) alanine-aminotransferase (ALT);
d) aspartate aminotransferase (AST);
e) Total fasting cholesterol;
f) fasting triglycerides; and
g) fasting glucose.

The instructions 110 to receive measurements of serum biomarkers do not include instructions to receive measurements of any one of alpha-2-macroglobulin, Apolipoprotein A1, Haptoglobin, or platelet count.

The instructions 111 to obtain average measurement of biomarkers of a population may include instructions to obtain average biomarkers for at least one of alpha-2-macroglobulin, Apolipoprotein A1, Haptoglobin, or platelet count.

The instructions 111 to obtain average measurements of biomarkers of a population may include instructions to identify a specific population to which the human subject belongs based on the received biographical information, and instructions to extract, from database(s) 109, average biomarker information for that specific population.

For example, the specific population may be identified based on the subject's sex, age, weight or BMI evaluation, nationality, ethnicity, area of residence, and/or medical history.

The inventors have surprisingly found that the average biomarker for the specific population are sufficiently accurate to identify the presence of liver conditions and/or to classify such liver conditions, despite the lack of subject specific information relating to these biomarkers. For example, the inventors surmise that the accuracy may be at least partially due to genetic dispositions that may be present in different ethnic groups, such that the ethnicity of a subject may be indicative of their risk of liver condition when certain biomarkers are evaluated. Similarly the inventors surmise that the accuracy may be at least partially due to risk factors that may be present in subjects of different sexes or BMI levels.

In some embodiments, system 100 further includes, or is functionally associated with, at least one input interface 130, such as a keyboard, touchscreen, touchpad, mouse, and the like, functionally associated with processor 106. A user, such as a medical practitioner or the subject, may use the input interface(s) 130 to provide at least some of the data received when executing instructions 110, such as at least some of the biographical information.

In some embodiments, system 100 further includes at least one transceiver 132, functionally associated with processor 106, and adapted for communication with the analyzer(s) 102, with database(s) 109, and/or with other devices adapted to provide the inputs received by execution of instructions 110 and/or 111, such as a scale.

In some embodiments, instructions 114 to identify the presence of a liver condition, and, if relevant, to classify the severity of the liver condition, further include instructions to provide an output including the identified presence and/or severity of the liver condition. In some such embodiments, system 100 further includes, or is functionally associated with, an output interface 134, such as a display screen or a speaker, via which the output is visually or audibly provided to a user, such as a medical practitioner. In some embodiments, the output may be provided in an electronic communication, such as an e-mail message, for example via transceiver 132.

Typically, a blood sample 140, used to extract the measurements of the serum biomarkers received system 100, is collected from the human subject using test tubes 142 as known in the art. In some embodiments, the blood sample must have sufficient volume to provide a 500 microliter volume of plasma or serum, after centrifugation thereof. In some embodiments, the blood sample must have sufficient volume to provide a 200 microliter volume of plasma or serum, after centrifugation thereof.

In some embodiments, at least one test tube 142 is a test tube containing lithium heparin or gel (SST). In some embodiments, at least one test tube 142 is a test tube containing a glycolytic inhibitor, such as sodium fluoride or potassium oxalate, required for extracting a fasting glucose biomarker measurement.

In some embodiments, the analyzer(s) 102 include an analyzers adapted to extract at least one GGT measurements, total bilirubin measurements, and/or ALT measurements. Examples of suitable analyzers include, Siemens Healthcare Diagnostics models Dimension-RXL, ArX, XPAND Vista, Atelica® CH 930, or Advia 1650, commercially available from Siemens® AG of Munich, Germany; models AU400, AU600, AU640, or AU2700 commercially available from Beckman Coulter® of Indianapolis, Indiana, USA; models T20, T20XT, T30, T60, and T60 new generation commercially available from Thermo Fisher Scientific® of Waltham, MA, USA; and Hitachi 917, Modular P, Integra 400, Cobas 6000, or Cobas 8000 commercially available from Roche Diagnostics® of Basel, Switzerland.

Portable diagnostic analyzer to offer a full complement: of CLIA-waived blood chemistry tests at the point of care. [Piccolo Xpress from Abbott Diagnostics]

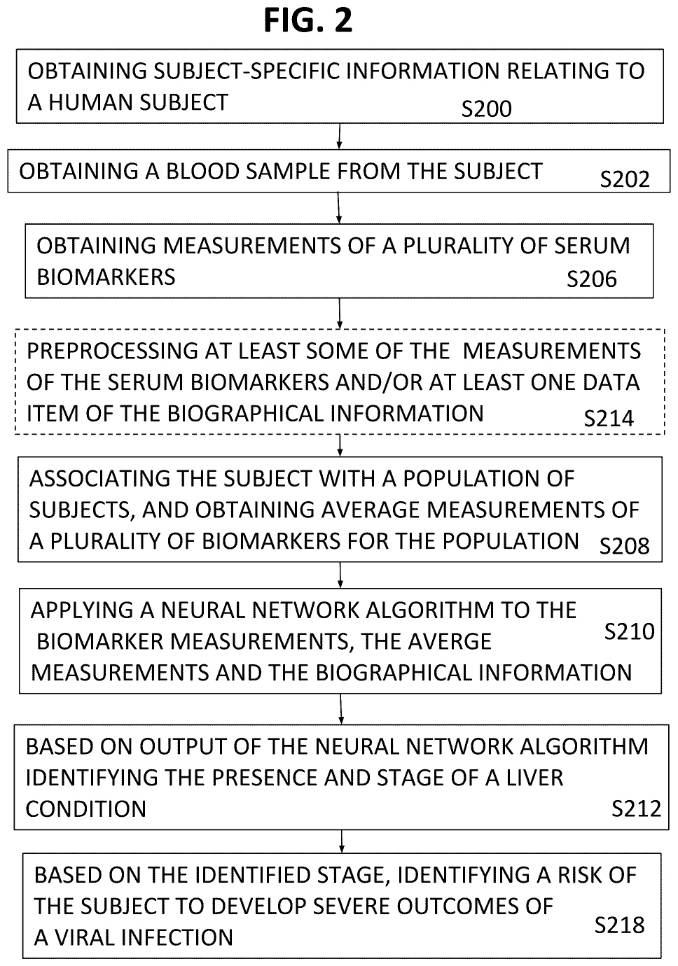

Reference is now additionally made to FIG. 2, which is a flow-chart of a method for identifying the presence of a liver condition, and classifying a severity thereof, according to an embodiment of the disclosed technology. The method of FIG. 2 is described herein with respect to the system of FIG. 1. However, any other suitable system may be used to implement the method of FIG. 2.

As seen in FIG. 2, at step S200, subject-specific information relating to a human subject, such as subject 10 of FIG. 1, is obtained, for example by processor 106 executing instructions 110. In some embodiments, the information includes biographical information, such as the subject's age, gender, height, and weight. In some embodiments, the obtained information includes anthropometric information, such as the subject's ethnicity, nationality, area of residence, and the like. In some embodiments, the obtained information includes medical information, such as information relating to the subject's previously diagnosed conditions, medications taken by the subject, and the like.

At step S202, which may occur before, after, or concurrently with step S200, a blood sample of the human subject is obtained. As discussed hereinabove with respect to FIG. 1, the blood sample is typically obtained in test tubes, using methods known in the art. In some embodiments, specific types of test tubes are used, as discussed hereinabove with respect to FIG. 1. Serum or plasma is then extracted from the blood sample, using methods known in the art, such as centrifugation.

In some embodiments, the obtained blood sample may optionally be diluted, or otherwise preprocessed. This may occur when the blood sample includes components which would interfere with the remainder of the method, such as lipids.

At step S206, measurements of a plurality of serum biomarkers are obtained from the serum or plasma in the blood sample, for example by processor 106 executing instructions 110. The measurements are computed by one or more analyzers, such as analyzers 102 of FIG. 1, and are received from the analyzer(s). The obtained measurements include measurements for at least three, at least four, and preferably all of:
a) total Bilirubin;
b) gamma-glutamyl transpeptidase (GGT);
c) alanine-aminotransferase (ALT);
d) aspartate aminotransferase (AST);
e) Total fasting cholesterol;
f) fasting triglycerides; and
g) fasting glucose.

At step S208, and based on the subject-specific information obtained at step S200, the subject is associated with a population of similar people, and average measurements of a plurality of biomarkers are obtained for the population, for example by processor 106 executing instructions 111, from a database such as database 109. The population may be based on one or more of the subject's age, sex, BMI value, nationality, ethnicity, area of residence, and known medical history. Step S208 occurs after step S200, but may occur before, after, or concurrently with steps S204 and S206.

The average measurements include average or standardized according to anthropometrics measurements of at least one of, and typically all of, alpha-2-macroglobulin, Apolipoprotein A1, Haptoglobin, or platelets count. Typically, measurements for these three biomarkers are not obtained from the plasma or blood of the subject, at step S206.

At step S210, a neural network algorithm is applied, for example by processor 106 executing instructions 112, to (i) at least some of, or all of, the measurements of the plurality of biomarkers; (ii) at least some of, or all of, the average measurements of biomarkers of the population; and (iii) at least some of, or to all of, the obtained user-specific information. Based on the output of the neural network algorithm, at step S210, the presence or absence of a liver condition is identified, and, in cases in which such liver condition is identified, its severity is classified, for example by processor 106 executing instructions 114.

In some embodiments, as part of identification of a liver condition at step S212, the subject is identified as belonging to one of four categories, each representing a class of liver conditions. In some embodiments, the four categories may include:
N0—no presumed liver fibrosis or steatosis;
N1—presumed steatosis only;
N2—presumed mild to moderate fibrosis; presumed NASH without severe fibrosis;
N3—presumed bridging fibrosis/cirrhosis; presumed NASH with severe fibrosis.

In some embodiments at step S214, which occurs prior to application of the neural network algorithm at step S210, at least some of the serum biomarker measurements and/or some subject-specific information items are preprocessed, for example by processor 106 executing instructions 118. In some such embodiments, the values used for the neural network algorithm, at step S210, are the preprocessed values resulting from step S214. In some such embodiments, the preprocessing includes logarithmically scaling at least some of the serum biomarker measurements, for example using logarithmic base 10. In some embodiments, the preprocessing includes standardizing values of at least some of the serum biomarker measurements and/or of at least some subject-specific information items.

In some embodiments, scaling at step S214 may include logarithmically scaling measurements of one or more of total bilirubin, GGT, ALT, AST, total cholesterol, fasting glucose, and triglycerides.

In some embodiments, standardizing at step S214 may include standardizing of age, total bilirubin measurement, GGT measurement, and ALT measurement, AST measurement, BMI, fasting blood glucose measurement, triglycerides measurement, and total cholesterol measurement.

In some embodiments, an output may be provided, for example via transceiver 132 or via output interface 134, the output including the identified presence and/or severity of the liver condition.

In some embodiments, at step S218 and based on the stage of the liver condition identified at step S212, a risk of the subject developing severe outcomes of viral infections, such as COVID-19, is identified, for example by processor 106 executing instructions 120.

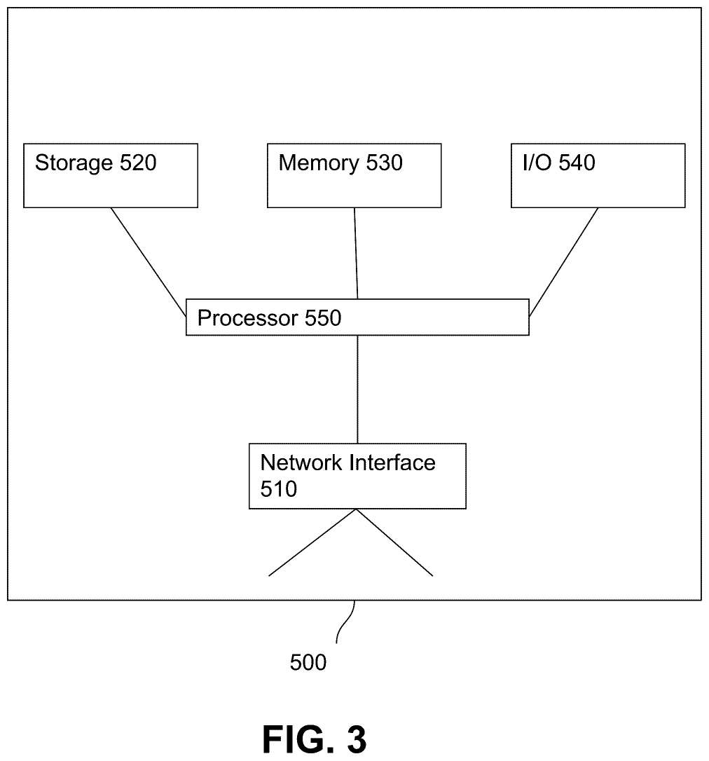

FIG. 3 shows a high-level block diagram of a device that may be used to carry out the disclosed technology. Device 500 comprises a processor 550 that controls the overall operation of the computer by executing the device's program instructions which define such operation. The device's program instructions may be stored in a storage device 520 (e.g., magnetic disk, database) and loaded into memory 530 when execution of the console's program instructions is desired. Thus, the device's operation will be defined by the device's program instructions stored in memory 530 and/or storage 520, and the console will be controlled by processor 550 executing the console's program instructions. A device 500 also includes one or a plurality of input network interfaces for communicating with other devices via a network (e.g., the internet). The device 500 further includes an electrical input interface. A device 500 also includes one or more output network interfaces 510 for communicating with other devices. Device 500 also includes input/output 540 representing devices which allow for user interaction with a computer (e.g., display, keyboard, mouse, speakers, buttons, etc.). One skilled in the art will recognize that an implementation of an actual device will contain other components as well, and that FIG. 3 is a high-level representation of some of the components of such a device for illustrative purposes. It should also be understood by one skilled in the art that the method and devices depicted in FIGS. 1 and 2 may be implemented using a device such as is shown in FIG. 3.

### EXAMPLES

Reference is now made to the following example, which, together with the above description, illustrates an embodiment of the invention in a non-limiting fashion.

### Example 1

A study which included 366 participating adult subjects diagnosed with COVID-19 was conducted. The subjects ranged from early-stage asymptomatic disease through late severe stage disease.

Liver conditions in the subjects were identified and characterized using the method of FIG. 2, such that the subjects were categorized into four categories, as seen in Table 1.

TABLE 1

| Classification | Meaning | % |
|---|---|---|
| Stage N0 | No presumed fibrosis or steatosis | 16.4% |
| Stage N1 | Presumed steatosis only | 53.0% |
| Stage N2 | Presumed fibrosis (not severe) | 25.4% |
| Stage N3 | Presumed severe fibrosis | 5.2% |

The prevalence of severe outcome of COVID-19 in subjects classified by the method of FIG. 2 into different categories was determined, as shown in Table 2.

TABLE 2

| | | Cox Mantel probability |
|---|---|---|
| Classification | Severe outcome prevalence | level |
| Stage N0 + N1 | 7.4% | P < 0.01 |
| Stage N2 + N3 | 17.4% | |

The results of the study demonstrate that subjects classified by the method of FIG. 2 as having liver fibrosis (stages N2 and N3) had a higher risk of developing severe outcomes of COVID-19 when compared with subjects classified as not having liver fibrosis (stages N0 and N1).

As such, the method of classifying liver conditions described hereinabove with respect to FIG. 2 may also be used by medical personnel to evaluate the risk level of a certain subject to develop severe outcomes of viral diseases, such as COVID-19. Thus, such classification may enable the medical personnel to treat the subject sooner, and in a better manner, to ensure their survival and recovery.

For the purposes of this disclosure, severe outcomes of COVID-19 include one or more of the following: need of invasive oxygen therapy such as tracheal intubation, need of prone ventilation, need of ECMO, need of renal replacement therapy, ICU transfer inside the hospital, need of inotrope or dopamine therapy, cardiac arrest, or palliative discharge.

For purposes of this disclosure, the term “substantially” is defined as “at least 95% of” the term which it modifies.

Any device or aspect of the technology can “comprise” or “consist of” the item it modifies, whether explicitly written as such or otherwise.

When the term “or” is used, it creates a group which has within either term being connected by the conjunction as well as both terms being connected by the conjunction.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

1. A method of identifying and classifying a liver condition in a human subject, the method comprising:
obtaining subject-specific information relating to the human subject, the subject-specific information including at least age, sex, height, and weight;
using a plurality of analyzers, obtaining from serum or plasma in a blood sample obtained from the human subject measurements of a plurality of serum biomarkers, the plurality of serum biomarkers including at least three biomarkers selected from the group consisting of total Bilirubin, gamma-glutamyl transpeptidase (GGT), alanine-aminotransferase (ALT), aspartate aminotransferase (AST), Total fasting cholesterol, fasting triglycerides, and fasting glucose;
based on the obtained subject-specific information, identifying a population to which the human subject belongs;
obtaining, from a database, average measurements of a second plurality of biomarkers for the population, the second plurality of biomarkers selected from the second group consisting of Alpha-2-Macroglobulin, Apolipoprotein A1, Haptoglobin and platelets count,
using a processor executing instructions stored in a non-transitory computer memory, applying a neural network algorithm to the measurements of the plurality of biomarkers, the average measurements, and the subject-specific information; and
based on an output of the neural network algorithm, identifying the presence of a liver condition and classifying a severity of the liver condition.

2. The method of claim 1, wherein the plurality of serum biomarkers excludes Alpha-2-Macroglobulin, Apolipoprotein A1, Haptoglobin and platelets count.

3. The method of claim 1, wherein at least one test tube used for obtaining the blood sample comprises at least one of a test tube containing lithium heparin, a test tube containing a glycolytic inhibitor, a test tube containing sodium fluoride, and a test tube containing potassium oxalate.

4. The method of claim 1, wherein the output of the neural network algorithm includes a classification into one of a plurality of stages, each stage indicating the presence or absence of the liver condition, and the severity of the liver condition.

5. The method of claim 1, further comprising, prior to the applying the neural network algorithm, pre-processing at least some of the measurements of the plurality of serum biomarkers or at least one data item of the subject-specific information.

6. The method of claim 5, wherein the pre-processing comprises logarithmically scaling at least some of the measurements of the plurality of serum biomarkers.

7. The method of claim 5, wherein the pre-processing comprises standardizing at least some of the measurements of the plurality of serum biomarkers and at least one data item of the biographical information.

8. The method of claim 1, wherein the obtained subject-specific information additionally includes at least one of nationality, ethnicity, area of residence, and medical history of the subject.

9. The method of claim 1, further comprising, based on the classification of the liver condition, evaluating the risk of the subject developing a severe outcome to a viral infection.

10. The method of claim 9, wherein the viral infection comprises a COVID-19 infection.

11. A system of identifying and classifying a liver condition in a human subject based on a blood sample obtained from the human subject, the system being functionally associated with at least one analyzer for analyzing the blood sample, the system comprising:
at least one of an input interface or a transceiver;
a database storing anthropometric and/or medical data relating to a plurality of subjects;
one or more processors functionally associated with the at least one input interface or transceiver and with the database; and
a non-transitory computer readable storage medium for instructions execution by the one or more processors, the non-transitory computer readable storage medium having stored:
instructions to receive subject-specific information relating to the human subject, the subject-specific information including at least age, gender, height, and weight;
instructions to receive, from the at least one analyzer, measurements of a plurality of serum biomarkers, the plurality of serum biomarkers including at least three biomarkers selected from the group consisting of total Bilirubin, gamma-glutamyl transpeptidase (GGT), alanine-aminotransferase (ALT), aspartate aminotransferase (AST), Total fasting cholesterol, fasting triglycerides, and fasting glucose;
instructions to identify, based on the received subject-specific information, a population to which the human subject belongs;
instructions to obtain from the database average measurements of a second plurality of biomarkers for the population, the second plurality of biomarkers selected from the second group consisting of Alpha-2-Macroglobulin, Apolipoprotein A1, Haptoglobin and platelets count
instructions to apply a neural network algorithm to the measurements of the plurality of biomarkers, the average measurements, and the subject-specific information; and
instructions to identify, based on an output of the neural network algorithm, the presence of a liver condition, and to classify a severity of the liver condition.

12. The system of claim 11, wherein the plurality of serum biomarkers excludes Alpha-2-Macroglobulin, Apolipoprotein A1, and Haptoglobin and platelets count.

13. The system of claim 11, wherein the non-transitory computer readable storage medium further has stored instructions, to be executed prior to execution of the instructions to applying the neural network algorithm, to pre-process at least some of the measurements of the plurality of serum biomarkers or at least one data item of the subject-specific information.

14. The system of claim 13, wherein the instructions to pre-process comprise instructions to logarithmically scale at least some of the measurements of the plurality of serum biomarkers.

15. The system of claim 13, wherein the instructions to pre-process comprise instructions to standardize at least some of the measurements of the plurality of serum biomarkers and at least one data item of the subject-specific information.

16. The system of claim 11, wherein the instructions to identify the presence of a liver condition and to classify the severity of the liver condition comprise instruction to classify the subject into one of a plurality of stages, each stage indicating the presence or absence of the liver condition, and the severity of the liver condition.

17. The system of claim 11, wherein the subject-specific information additionally includes at least one of nationality, ethnicity, area of residence, and medical history of the subject.

18. The system of claim 11, wherein the non-transitory computer readable storage medium further has stored instructions to evaluate the risk of the subject developing a severe outcome to a viral infection based on the classification of the liver condition.
